(19) United States
(12) Patent Application Publication
(10) Pub. No.: US 2008/0138350 A1
(43) Pub. Date: Jun. 12, 2008

(54) PROCESS FOR USE OF FLUOROQUINOLONES TO REDUCE OR MODULATE INFLAMMATION DUE TO EYE DISEASE OR OPHTHALMIC SURGERY

(76) Inventor: Michael D. Bennett, Honolulu, HI (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/876,435

(22) Filed: Oct. 22, 2007

Related U.S. Application Data
(60) Provisional application No. 60/853,155, filed on Oct. 20, 2006.
(60) Provisional application No. 60/919,493, filed on Mar. 22, 2007.

(51) Int. Cl. A61K 31/4709; A61K 31/47; A61K 31/7088; A61K 39/395; A61P 27/02

(52) U.S. Cl. 424/145.1; 514/311; 514/314; 514/44

(57) ABSTRACT

The present invention provides a safer and more effective way to treat ophthalmic disease and to perform ophthalmic surgeries while reducing the risk of vision loss due to the inflammatory response in the eye. The invention generally relates to the use of fluoroquinolones to reduce inflammation due to ophthalmic disease or ophthalmic surgery, including treatments for macular degeneration, diabetes, and vascular ischemic diseases, and other ophthalmic diseases. One aspect of the present invention involves using an anti-VEGF medication along with moxifloxacin in treating ophthalmic disease and in reducing inflammatory response in the eye following surgery for treating diseases like wet macular degeneration. Another aspect of the present invention involves delivering a fluoroquinolone combination to the eye in a sustained delivery application or device.

### CROSS-REFERENCES TO RELATED APPLICATIONS

Priority is claimed to U.S. Provisional Application Ser. No. 60/853,155, filed on Oct. 20, 2006, the contents of which are incorporated by reference in their entirety, and to U.S. Provisional Application Serial No. 60/919,493, filed on Mar. 22, 2007, the contents of which are incorporated by reference in their entirety.

### FIELD OF THE INVENTION

The present invention relates generally to the use of fluoroquinolones to reduce or modulate inflammation due to eye disease or ophthalmic surgery.

### BACKGROUND OF THE INVENTION

Intravitreal injections are currently approved for treating wet macular degeneration. But intravitreal injections have significant risks, including endophthalmitis, which can be more devastating to vision than wet macular degeneration itself. Patients with wet macular degeneration are often older patients dependent on others for transportation and may have a decreased ability for follow-up and self-care. The frequency of intravitreal injections requires diligence with topical antibiotics in the periprocedure period.

Fluoroquinolones are broad-spectrum antibiotics effective against gram-positive and gram-negative bacteria. Moxifloxacin is a preservative-free preparation that has been shown to be non-toxic intracamerally in a rabbit model at a concentration of 160 μg/mL. Doctors have also used moxifloxacin intracamerally in more than one thousand human eyes with minimal toxicity. Pegaptanib combined with moxifloxacin appears to have no significant photoreceptor toxicity in the short term.

It is known that fluoroquinolones, in addition to killing bacteria, can also be used to reduce inflammation in the body. But fluoroquinolones have not been used to reduce or modulate inflammation due to macular degeneration, diabetes, vascular ischemic and other ophthalmic diseases, or ophthalmic surgery, including vitreo-retinal surgery. Doctors have used other anti-inflammatory drugs to reduce inflammation in the eye after surgery, but usually on an as-needed basis after inflammation has developed. As a result, the inflammatory response is sometimes a significant cause of visual loss and, if treated too late, the patient may suffer permanent visual loss.

Inflammation has been associated with ophthalmic diseases, including macular degeneration, and occurs following ophthalmic surgery. In these conditions, macrophages play a significant role in the inflammatory process. Macrophages are found in all forms of inflammation and have been identified in surgical CNV membranes in patients with macular degeneration. The body recruits macrophages to clean up tissue debris, and in response the macrophages secrete cytokines, including VEGF and TNF-α. TNF-α can induce choroidal endothelial cells to secrete angiogenic factors, including VEGF. Pathologic ocular neovascularization can be found in diabetes, macular disease, and ischemia as is seen following vascular occlusion. This neovascular process has been found to be inhibited in genetically transformed mice that are deficient in TNF-α. Macrophages can potentiate inflammation and promote abnormal neovascularization by secretion of VEGF or through induced secretion of VEGF, mediated by macrophage-produced TNF-α. Fluoroquinolones are taken up by macrophages and are accumulated intracellularly on average 3- to 12-fold depending on the cell type and the fluoroquinolone used. This intracellular accumulation can cause an antiproliferative effect and can reduce inflammation through its immunomodulation.

It should be appreciated that there exists a need for safer and more effective synergistic ways to treat macular degeneration and other ophthalmic diseases, and to perform ophthalmic surgeries, including vitreo-retinal surgeries, while reducing the risk of vision loss due to inflammation in the eye. It should also be appreciated that there exists a need to determine optimal dosages, dosage ranges, and dosage procedures for administering fluoroquinolones within the eye. The present invention fulfills these needs and provides further related advantages.

### SUMMARY OF THE INVENTION

The present invention provides a safer and more effective way to treat ophthalmic disease and to perform ophthalmic surgeries while reducing the risk of vision loss due to the inflammatory response in the eye. The invention generally relates to the use of fluoroquinolones to reduce inflammation due to ophthalmic disease or ophthalmic surgery, including treatments for macular degeneration, diabetes, and vascular ischemic diseases, and other ophthalmic diseases. One aspect of the present invention involves using an anti-VEGF medication along with moxifloxacin in treating ophthalmic disease and in reducing inflammatory response in the eye following surgery for treating diseases like wet macular degeneration. Another aspect of the present invention involves delivering a fluoroquinolone or fluoroquinolone combination to the eye in a sustained delivery application or device.

### BRIEF DESCRIPTION OF THE DRAWINGS

FIG. 1 is a chart showing the mean change in visual acuity over time for eighty eyes treated with a combination of pegaptanib and moxifloxacin in accordance with an embodiment of the present invention.

FIG. 2 is a chart showing the mean change in visual acuity over time for sixteen eyes treated with a combination of pegaptanib and moxifloxacin in accordance with an embodiment of the present invention. These eyes received pegaptanib/moxifloxacin combination therapy only, with a history of no previous or adjuvant treatments.

### DETAILED DESCRIPTION OF THE INVENTION

In view of the foregoing, it is an object of the present invention to provide a safer and more effective way to treat ophthalmic disease and perform ophthalmic surgeries while reducing the risk of vision loss due to inflammation in the eye. The present invention generally relates to the use of fluoroquinolones to reduce inflammation due to macular degeneration, diabetes, vascular ischemic and other ophthalmic diseases, and ophthalmic surgery.

These ophthalmic conditions are multifactorial in their pathophysiology. To date, the treatment of macular degeneration and other vascular ischemic conditions has been directed at a single target—anti-vascular derived growth factor. This approach usually requires multiple injections and long-term administration. In the present invention, combining multiple forms of treatment addresses the various disease states at multiple different therapeutic points along their inflammatory cascade.

Moreover, in the present invention, sustained delivery and the combination of medications results in smaller drug dosing and reduces the frequency of needed administration. The combination of medications in the present invention also has an added potential synergistic effect.

Fluoroquinolones modulate inflammation in the presence of a co-stimulant. Thus, targeting combination medications enhances the body's response to treatment. For example, in the present invention, fluoroquinolones help control and minimize cellular damage when combined with different forms of treatment, such as anti-vascular derived growth factor (VEGF), VEGF trap, Ciliary Neutrophic Factor (CNTF), anti-platelet derived growth factor (PDGF), cyclooxygenase inhibitors, intraocular pressure lowering medications, steroids (such as Kenalog), and other neuroprotective and anti-inflammatory agents.

Eye drops and intravitreal injections are the conventional dosage forms that account for greater than ninety percent of the currently available ophthalmic formulations. Despite good acceptance by patients, the conventional dosage forms pose major problems ranging from rapid pre-corneal drug loss reducing the drug bioavailability to complications associated with repeated intraocular injections. To improve bioavailability and reduce the complications associated with repeated injections, the present invention uses sustained delivery of fluoroquinolones alone or in combination with other medications. The sustained delivery in the present invention can be achieved through a number of different delivery systems, including but not limited to polymeric gels, colloidal systems including liposomes and nanoparticles, cyclodextrins, collagen shields, diffusion chambers, flexible hydrophobic polytetrafluoroethylene carrier strips, and intravitreal implants, such as the Retisert implant marketed by Bausch & Lomb Inc.

In the present invention, ocular and intraocular drug delivery systems deliver these sight-saving fluoroquinolone combinations to the back of the eye. These drug delivery systems include: using the sclera itself as a drug delivery reservoir, “prodrug” formulations that pass through the tissue, tiny biodegradable pellets that release the combinations over time, intravitreal implants, intravitreal silicone inserts, intravitreal and transscleral poly(lactic-co-glycolic acid) microspheres, calcium-alginate inserts, encapsulated cells, transscleral iontophoresis, nanoparticles (e.g., calcium phosphate), and genetically modified viruses that can deliver therapeutic proteins into therapy.

One aspect of the present invention involves using an anti-VEGF medication, such as pegaptanib (trade name Macugen), ranibizumab (trade name Lucentis), or bevacizumab (trade name Avastin), along with moxifloxacin, a fluoroquinolone, in treating wet macular degeneration and reducing inflammation in the eye for wet macular degeneration. The use of pegaptanib along with moxifloxacin was tested in ninety eyes of seventy-five patents who gave their informed consent. Although the study described below was conducted using a fluoroquinolone (moxifloxacin) in combination with an anti-VEGF medication (pegaptanib), the present invention encompasses the use of fluoroquinolones in combination with other medications for treating ophthalmic disease, including macular degeneration, diabetes, and other ischemic diseases.

Patients were pre-treated starting three days before injection and for five days after injection with topical moxifloxacin. The intravitreal injection was administered under controlled aseptic conditions, using sterile gloves, a sterile drape, and a sterile lid speculum. The periocular area was cleaned with a povidone-iodine preparation.

The patients' eyes were injected with pegaptanib combined with approximately 165 μg of moxifloxacin (0.5% ophthalmic preservative-free solution) every six weeks. One drop (33 μL) of moxifloxacin was drawn into the pre-packaged pegaptanib (0.3 mg/90 μL) syringe.

The average pseudophakic human eye contains 5.0 mL of vitreous. An injection of 165 μg of moxifloxacin combined with pegaptanib results in a moxifloxacin concentration of 33 μg/mL in the vitreous cavity, which is thirteen times greater than the median MIC needed for fluoroquinolone resistant coagulase-negative Staphylococcus (2.5 μg/mL) and nineteen times greater than the MIC needed for resistant Staphylococcus aureus (1.75 μg/mL). Moxifloxacin has in vitro MICs of less than 0.20 μg/mL for most pathogens known to cause infectious endophthalmitis. Staphylococcus epidermidis and Staphylococcus aureus are the most common pathogens implicated in bacterial endophthalmitis. The half life of intravitreal moxifloxacin in rabbits has been measured at 1.72 hours; a 165 μg injection would maintain therapeutic levels at 12 hours.

Treatment was directed by clinical exam and response, targeting the vascular and extravascular components of macular degeneration. Visual acuity was assessed with ETDRS charts pre- and post-treatment. OCT, FA, and Microperimetry were performed when indicated. Patients were followed for an average of 14 months (range 9-16 months). The average patient age was 81 years, with a range of 56-96 years. Forty-eight percent (36) patients were male and 52 percent (39) patients were female.

All patients with new lesions and previously treated lesions (PDT, IVK, and STK) that were still active were enrolled in the study. All lesions, regardless of size (100μ-8DD) or type (classic or occult neovascular membrane), were enrolled in the study.

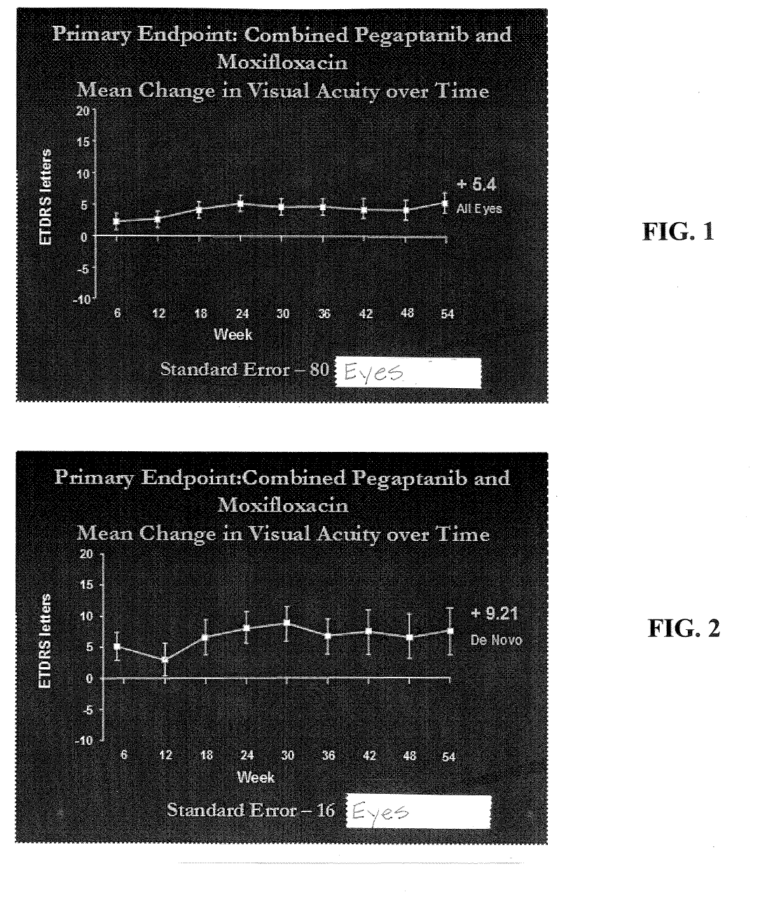

Of the ninety eyes, nine (10 percent) lost two to six lines of vision. Eighty-one eyes (90 percent) lost less than two lines of vision. Seventy eyes (77.8 percent) had stable or improved vision. Forty-two eyes (46.7 percent) gained five letters (one line) or more. Fifteen eyes (16.7 percent) gained ten letters (two lines) or more. Nine eyes (10 percent) gained fifteen letters (three lines) or more. FIG. 1 is a chart showing the mean change in visual acuity over time for eighty of these eyes. Ten eyes dropped out of the study before it was completed.

Of the ninety eyes, twenty-one eyes (23.3 percent) received pegaptanib/moxifloxacin combination therapy only, with a history of no previous or adjuvant treatments. This subset included all lesions, regardless of size (100μ-8DD) or type (classic or occult neovascular membrane), without previous or adjuvant PDT, IVK, or STK. Of this twenty-one eye subset, one eye (5.8 percent) lost three lines of vision. Twenty eyes (95.2 percent) lost less than two lines of vision. Nineteen eyes (90.5 percent) had stable or improved vision. Twelve eyes (57.1 percent) gained five letters (one line) or more. Six eyes (28.5 percent) gained ten letters (two lines) or more. Two eyes (9.5 percent) gained fifteen letters (three lines) or more. FIG. 2 is a chart showing the mean change in visual acuity over time for sixteen of these eyes. Five of these eyes dropped out of the study before it was completed.

Of the ninety eyes, sixty-nine eyes (76.7 percent) had been treated or had adjuvant treatment with various modalities, including photodynamic therapy, intravitreal kenalog injection, and subtenon kenalog injection. Of this sixty-nine eye subset, eight eyes (11.6 percent) lost two to six lines of vision. Sixty-one eyes (88.4 percent) lost less than two lines of vision. Fifty-one eyes (73.9 percent) had stable or improved vision. Thirty eyes (43.5 percent) gained five letters (one line) or more. Nine eyes (13.0 percent) gained ten letters (two lines) or more. Seven eyes (10.1 percent) gained fifteen letters (three lines) or more.

Based upon the study described above, it was found that pegaptanib combined with moxifloxacin is effective in treating wet macular degeneration while reducing inflammation in the eye after surgery. A preferred dose of 165 μg of moxifloxacin every six weeks was established, although a dose in the range from 0.1 μg to 320 μg every six weeks would provide for inflammation reduction without being toxic. The total number of doses would depend upon the individual case. Some cases would require only a single dose, while other cases would require doses to be administered for the remainder of the patient's life. Doses could also be administered in a sustained release format. In the study, the doses were injected intravitreally, although the doses could also be administered elsewhere in the eye, next to the eye in the sub tenon's capsule or sub conjunctival space, orally, or intravenously. Intracameral injection of antibiotics into the anterior chamber is possible, although suboptimal for preventing endophthalmitis if bacteria compromise the vitreous.

Although the study described above used moxifloxacin, the present invention encompasses the use of other fluoroquinolones in combination with other medications and agents to modulate and reduce inflammation due to ophthalmic surgery and ophthalmic diseases, such as macular degeneration, diabetes, and vascular ischemic diseases.

The present invention has been described above in terms of presently preferred embodiments so that an understanding of the present invention can be conveyed. However, there are other embodiments not specifically described herein for which the present invention is applicable. Therefore, the present invention should not to be seen as limited to the forms shown, which is to be considered illustrative rather than restrictive.

I claim:

1. A process for using a fluoroquinolone to reduce or modulate inflammation in an eye due to eye disease or ophthalmic surgery, the process comprising the steps of:
combining a predetermined amount of the fluoroquinolone with a predetermined amount of an anti-VEGF medication to make a fluoroquinolone/anti-VEGF mixture; and
administering the fluoroquinolone/anti-VEGF mixture to the eye.

2. The process of claim 1, wherein the fluoroquinolone is moxifloxacin.

3. The process of claim 2, wherein the fluoroquinolone/anti-VEGF mixture contains between approximately 0.1 μg and approximately 320 μg of moxifloxacin.

4. The process of claim 3, wherein the fluoroquinolone/anti-VEGF mixture contains approximately 165 μg of moxifloxacin.

5. The process of claim 1, wherein the anti-VEGF medication is selected from the group consisting of pegaptanib, ranibizumab, and bevacizumab.

6. The process of claim 5, wherein the anti-VEGF medication is pegaptanib.

7. The process of claim 6, wherein the fluoroquinolone/anti-VEGF mixture contains approximately 0.3 mg of pegaptanib.

8. The process of claim 1, wherein the fluoroquinolone/anti-VEGF mixture further comprises an agent selected from the group consisting of ciliary neutrophic factors, anti-platelet derived growth factors, cyclooxygenase inhibitors, intraocular pressure lowering medications, and steroids.

9. The process of claim 1, wherein the fluoroquinolone/anti-VEGF mixture provides sustained delivery of the fluoroquinolone and anti-VEGF medication to the eye.

10. The process of claim 1, further comprising the step of associating the fluoroquinolone/anti-VEGF mixture with a sustained delivery system selected from the group consisting of polymeric gels, colloidal systems, cyclodextrins, collagen shields, diffusion chambers, flexible hydrophobic polytetrafluoroethylene carrier strips, and intravitreal implants.

11. The process of claim 1, wherein the step of administering includes administering the fluoroquinolone/anti-VEGF mixture to a rear portion of the eye by means of a drug delivery system selected from the group consisting of prodrug formulations, biodegradable pellets, intravitreal implants, intravitreal silicone inserts, poly(lactic-co-glycolic acid) microspheres, calcium-alginate inserts, encapsulated cells, transscleral iontophoresis, nanoparticles, and genetically modified viruses.

12. The process of claim 1, wherein the step of administering includes administering the fluoroquinolone/anti-VEGF mixture to the eye by intravitreal injection.

13. A process for reducing or modulating inflammation in an eye due to eye disease or ophthalmic surgery, the process comprising the steps of:
preparing a plurality of doses of a medication to be administered to the eye, the medication comprising a fluoroquinolone and an anti-VEGF medication; and
administering the plurality of doses to the eye.

14. The process of claim 13, wherein the fluoroquinolone is moxifloxacin.

15. The process of claim 14, wherein each of the plurality of doses contains between approximately 0.1 μg and approximately 320 μg of moxifloxacin.

16. The process of claim 15, wherein each of the plurality of doses contains approximately 165 μg of moxifloxacin.

17. The process of claim 13, wherein the anti-VEGF medication is selected from the group consisting of pegaptanib, ranibizumab, and bevacizumab.

18. The process of claim 17, wherein the anti-VEGF medication is pegaptanib.

19. The process of claim 18, wherein each of the plurality of doses contains approximately 0.3 mg of pegaptanib.

20. The process of claim 13, wherein each of the plurality of doses further comprises an agent selected from the group consisting of ciliary neutrophic factors, anti-platelet derived growth factors, cyclooxygenase inhibitors, intraocular pressure lowering medications, and steroids.

21. The process of claim 13, wherein each of the plurality of doses provides sustained delivery of the fluoroquinolone and anti-VEGF medication to the eye.

22. The process of claim 13, further comprising the step of associating each of the plurality of doses with a sustained delivery system selected from the group consisting of polymeric gels, colloidal systems, cyclodextrins, collagen shields, diffusion chambers, flexible hydrophobic polytetrafluoroethylene carrier strips, and intravitreal implants.

23. The process of claim 13, wherein the step of administering includes administering each of the plurality of doses to a rear portion of the eye by means of a drug delivery system selected from the group consisting of prodrug formulations, biodegradable pellets, intravitreal implants, intravitreal silicone inserts, poly(lactic-co-glycolic acid) microspheres, calcium-alginate inserts, encapsulated cells, transscleral iontophoresis, nanoparticles, and genetically modified viruses.

24. The process of claim 13, wherein the step of administering includes administering each of the plurality of doses to the eye by intravitreal injection.

25. A process for using a fluoroquinolone to reduce or modulate inflammation in an eye due to eye disease or ophthalmic surgery, the process comprising the steps of:
associating the fluoroquinolone with a sustained delivery system selected from the group consisting of polymeric gels, colloidal systems, cyclodextrins, collagen shields, diffusion chambers, flexible hydrophobic polytetrafluoroethylene carrier strips, and intravitreal implants; and
administering the fluoroquinolone to the eye by means of the sustained delivery system.
